(19) United States
(12) Patent Application Publication
(10) Pub. No.: US 2005/0170356 A1
(43) Pub. Date: Aug. 4, 2005

(54) Multi-reagent pack

(76) Inventors: Fareed Kureshy, Del Mar, CA (US); Vijay K. Mahant, Murrieta, CA (US); Shailendra Singh, Carlsbad, CA (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 10/513,458

(22) PCT Filed: May 28, 2003
(86) PCT No.: PCT/US03/16903

Related U.S. Application Data
(60) Provisional application No. 60/383,896, filed on May 28, 2002.

(30) Foreign Application Priority Data
May 29, 2002 (WO) PCT/US02/17006

(51) Int. Cl. C12Q 1/68; G01N 33/53; C12M 1/34; H01L 29/08

(52) U.S. Cl. 435/6; 435/7.1; 435/287.2; 257/40

(57) ABSTRACT

A multi-reagent pack has a plurality of reagent compartments and further includes a read-write memory chip that carries data pertaining to a particular test protocol that employs reagents from the reagent compartments. Data may be transferred between the reagent pack and an analytic device, and optionally further to a supplier.

This application claims the benefit of U.S. provisional patent application with the Ser. No. 60/383,896, filed May 28, 2002, and international patent application with the serial number PCT/US02/17006, filed May 29, 2002, both of which are incorporated herein by reference.

### FIELD OF THE INVENTION

The field of the invention is storage devices, particularly as they relate to automated analytic devices.

### BACKGROUND OF THE INVENTION

Genomics and proteomics research made a vast number of nucleotide and peptide sequences available for analysis. Consequently, high-throughput screening of samples for the presence and/or quantity of a vast number of known genes or polypeptides has gained considerable interest in recent years. With the growing number of high throughput devices, reagent management has become increasingly important to reduce operator intervention during screening, and numerous configurations and methods for reagent management are known in the art.

For example, various high-throughput screening devices use liquid ports that receive reagents from externally mounted reservoirs via fluid lines. Externally mounted reservoirs advantageously increase the total volume of reagent that can be supplied to an analyzer and are generally only limited by the capacity of the reservoirs. However, while analyzers with such configurations can be operated for relatively long periods without operator intervention to refill the reservoir, externally mounted reservoirs are impracticable where the same analyzer is employed for multiple test procedures with varying reagent requirements.

In other examples, reagents may be pre-filled in standard multi-well plates that are then inserted into a robotic device. Such configurations typically overcome the problems associated with fast adaptability for multiple test procedures with varying reagent requirements. However, due to the size of the reservoirs in the multi-well plate, the available volume is relatively limited. Furthermore, such systems typically require multi-well plate handling capabilities. Moreover, the multi-well plates typically need to be labeled (in most cases via bar code) to avoid improper use.

Barcode labeling is relatively simple and therefore commonly used in high-throughput screening. However, and especially where fluids are handled, bar codes may be contaminated, or mechanically degraded where the container is repeatedly used. To overcome such problems, Konrad describes in his published U.S. patent application with the serial No. 2001/0021356 a test tube that comprises a microchip (in the cap and/or in embedded in the test tube). An operator then uploads data (e.g., patient name or analysis results) using a computer onto the microchip, preferably via a speech recognition program. While such configurations solve at least some of the problems associated with bar code labels, various disadvantages nevertheless remain. Among other things, correct selection and uploading of the data is still bound to an operator, which will likely result in a misoperation. Furthermore, an operator still needs to ascertain that the test tube is used in the proper test procedure. Therefore, the operator will need to read the information on the chip before inserting the test tube into the appropriate analyzer. Still further, the data on the chip remains static until the operator updates the data stored on the device, thereby significantly increasing operator intervention.

Thus, although various systems for reagent containers are known in the art, numerous problems still remain. Therefore, there is still a need for an improved methods and systems for reagent handling, and especially reagent handling for automated analytic devices.

### SUMMARY OF THE INVENTION

The present invention is directed to configurations and methods for a multi-reagent pack in which a read-write memory chip provides and receives data from an analytic device, wherein the data are modified by the analytic device over the course of multiple test procedures. Modified data are preferably used to alert a user of a particular condition, to prevent unauthorized use/refill of the multi-reagent pack, and are further optionally transferred to a supplier via that analytic device.

In one aspect of the inventive subject matter, a disposable multi-reagent pack has a housing that includes a first compartment and a second compartment, wherein the first and second compartments include a first pre-filled reagent and a second pre-filled reagent, respectively. It is further preferred that such devices include a closing element that is coupled to the housing and movable between a first and a second position, wherein the closing element is moved between the first and second position by an actuator when the multi-reagent pack is disposed within the analytic device, and wherein at least one of the first and second compartments is accessible to a pipette when the closing element is in the second position. A read-write memory chip provides to the analytic device at least one of a multi-reagent pack specific information, a reagent-specific information, a test-specific information, a locking code and a chronologic information, and the analytic device provides to the read-write memory chip at least one of a reagent-specific information, a locking code, and a chronologic information.

Particularly contemplated multi-reagent pack specific information includes an individual identifier, a manufacture date of the multi-reagent pack, a list of test procedures available for the first and second pre-filled reagents, an environmental parameter, and/or a shelf-life of the first and/or second pre-filled reagents. Particularly preferred reagent-specific information includes the chemical composition, fill date, original fill volume, and/or positional information of the first and second pre-filled reagents, and/or the remaining volume of the first and/or second pre-filled reagents.

Similarly, preferred test-specific information comprises a test procedure that uses at least one of the first and second pre-filled reagents, a maximum allowed number of test procedures that uses the multi-reagent pack, calibration data, and/or a locking code, while preferred chronologic information comprises cumulative time during which the closing element was in the second position, or time elapsed since first use of the multi-reagent pack. Preferred locking codes may prohibit further use of the multi-reagent pack or can be erased using a user password.

Contemplated devices may further include a data transfer interface electronically coupled to a supplier that receives data from at least one of the read-write memory chip and the analytic device, wherein such data may be employed to initiate delivery of another multi-reagent pack or to forecast demand for at least one of the first and second pre-filled reagents. With respect to the reagents, it should be recognized that various reagents may be used in the multi-reagent pack, and suitable pre-filled reagents include spectroscopically detectable agents, fluorometrically detectable agents, luminometrically detectable agents, radiometrically detectable agents, nucleic acids, polypeptides, and/or a buffer.

In another aspect of the inventive subject matter, a multi-reagent pack has a first pre-filled reagent and a second pre-filled reagent, and further includes a read-write memory chip that provides calculated volume information of the first and second pre-filled reagents to an analytic device, wherein the calculated volume information is generated by the analytic device using previous consumption of the first and second pre-filled reagent of the multi-reagent pack in the analytic device, and wherein the calculated volume information is written by the analytic device onto the read-write memory chip.

Most preferably, the calculated volume information is computed by the analytic device using an initial volume information provided by the read-write memory chip, and the analytic device generates a locking code that prevents further use of the multi-reagent pack in the analytic device (e.g., locking code is written by the analytic device onto the read-write memory chip). Alternatively, or additionally, the analytic device may be programmed to perform a test procedure, wherein a validation sequence calculates reagent requirements for the first and second pre-filled reagents, and wherein the test procedure is not performed if the reagent requirements for the first and second pre-filled reagents are greater than the calculated volume information of the first and second pre-filled reagents. Where desired, contemplated analytic devices may further comprise a data transfer interface that is electronically coupled to a supplier that receives data from at least one of the read-write memory chip and the analytic device (e.g., to initiate delivery of another multi-reagent pack or to forecast demand for at least one of the first and second pre-filled reagents).

In a further aspect of the inventive subject matter, a multi-reagent pack has a first pre-filled reagent and a second pre-filled reagent, and further has a read-write memory chip that provides calibration data specific to at least one of the first and second pre-filled reagents. In such devices, it is preferred that the read-write memory chip further provides to the analytic device a protocol for a test procedure using the first and second pre-filled reagents, or an identifier that initiates a test procedure using the first and second pre-filled reagents. Alternatively, or additionally, such devices may further include a data transfer interface that is electronically coupled to a supplier, wherein the supplier provides a data upgrade to at least one of the read-write memory chip and the analytic device (e.g., corrected calibration data or a modified protocol for the test procedure using the first and second pre-filled reagents).

Various objects, features, aspects and advantages of the present invention will become more apparent from the following detailed description of preferred embodiments of the invention, along with the accompanying drawing in which like numerals represent like components.

### BRIEF DESCRIPTION OF THE DRAWING

FIG. 1 is a schematic view of an exemplary reagent pack.

FIG. 2 is a schematic view of an analytic device with a plurality of multi-reagent packs loaded.

### DETAILED DESCRIPTION

As used herein, the term “disposable multi-reagent pack” refers to a device that has at least two physically separated (e.g., via separate containers) reagents, wherein the operator of the device discards the device after a predetermined number of uses or after a minimum quantity of the reagents is reached. Alternatively, the user may also send the device to a supplier that then refills the device. Thus, and viewed from a different perspective, the disposable multi-reagent pack is not refilled by the operator after a predetermined number of uses or after a minimum quantity of the reagents is reached.

As further used herein, the term “pre-filled reagent” generally refers to a reagent that is filled in the multi-reagent pack by a person other than the operator, and most typically by a commercial supplier of the multi-reagent pack. Therefore, a patient sample (e.g., blood sample), buffer, or detection reagent that is filled into the multi-reagent pack by the operator or at the point of use (e.g., hospital or laboratory) is not considered a pre-filled reagent. The terms “commercial supplier” and “supplier” are used interchangeably herein and refer to an entity that sells at least one of the reagents, and most typical at least one of the reagents in the multi-reagent pack.

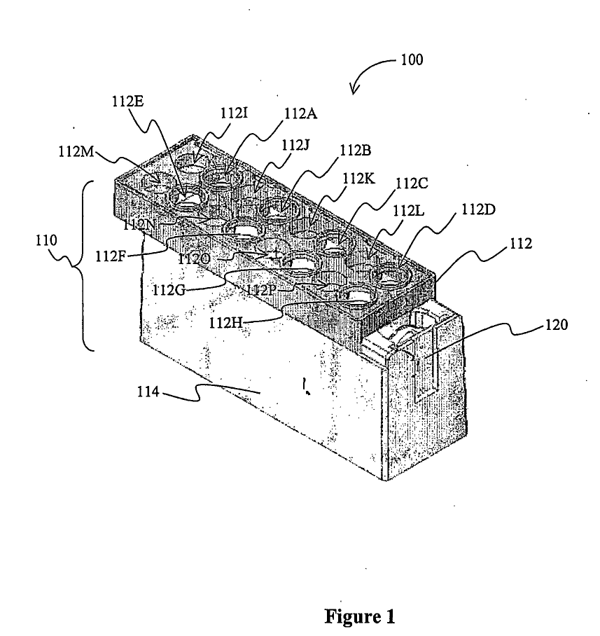

It is generally contemplated that improved multi-reagent packs include at least two, and more typically four to eight compartments within a housing, wherein the housing still further includes a read-write memory chip and a closing element. More particularly, and as shown in FIG. 1, an exemplary multi-reagent pack 100 has a housing 110 with a sliding lid cover 112 and a body 114 that includes reagent compartments (only partly visible through openings). The lid 112 has openings 112A-112H that provide access for a pipette (not shown) to the reagent compartments when the lid is in the open position. Seal elements 112I-112Q sealingly close the reagent compartments when the lid is in the closed position. A read-write memory chip (optionally coupled to a CPU and/or power supply) 120 is coupled to the body 114.

With respect to the dimensions and shape of the multi-reagent pack, it should be recognized that the particular size and shape will generally depend on a specific analytic device. For example, where relatively large reagent volumes and/or numerous reagent types are required, the size may be up to 1000 cm³ (and even higher). More typically, however, contemplated sized of the multi-reagent pack will be in the range of between about 50-500 cm³ (or less where appropriate). Similarly, it should be recognized that the shape of the particular reagent container need not be limited to a generally box-shaped configuration. For example, alternative container shapes especially include cylindrical shapes.

Therefore, the number, size, and volume of reagent compartments in contemplated multi-reagent packs may vary considerably, and it is generally contemplated that suitable multi-reagent packs may have between one and twenty, more typically between two and ten, and most typically between four and eight reagent compartments. Typically, the size and volume of the reagent compartments will be identical, but reagent compartments of differing size and volume are also contemplated. Furthermore, it should be appreciated that the arrangement of the reagent compartments is not critical to the inventive subject matter presented herein. Thus, while reagent compartments are typically sequentially arranged, alternative arrangements may be determined by alternative shapes of the body/and/or multi-reagent pack. Depending on the number, volume, and configuration of the reagent compartments, it should be appreciated that the shape of the body of contemplated multi-reagent packs will vary substantially. However, it is generally preferred that the body and closing element together have a box-shaped or cylindrical shape.

Consequently, the closing element need not be limited to a sliding lid cover, and all known manners of temporarily closing a container are deemed suitable so long as the closing element allows complete (and most preferably liquid-proof) closure of all of the compartments at one time, and opening of at least one container at another time such that at least part of the reagent in the container can be accessed by a liquid manipulation device (e.g., pipette, stirring device, sample-holder, etc). Therefore, and especially where the multi-reagent pack has a box shape, suitable closing elements include sliding lids (or other cover elements, including flexible/movable cover elements with an opening). On the other hand, where the multi-reagent pack has a cylindrical shape, it is contemplated that the closing element may also be a rotating cover with one or more openings that allows access to the reagent one or more containers at a time. Still further particularly contemplated closing elements will sealingly engage with the body and/or reagent compartments to allow shipping and transportation of the multi-reagent pack while reagents are present in the multi-reagent pack. Alternatively, however, it is also contemplated that at least one of the compartments has an additional removable closing cover for spill-proof enclosure of the reagent in the reagent compartment.

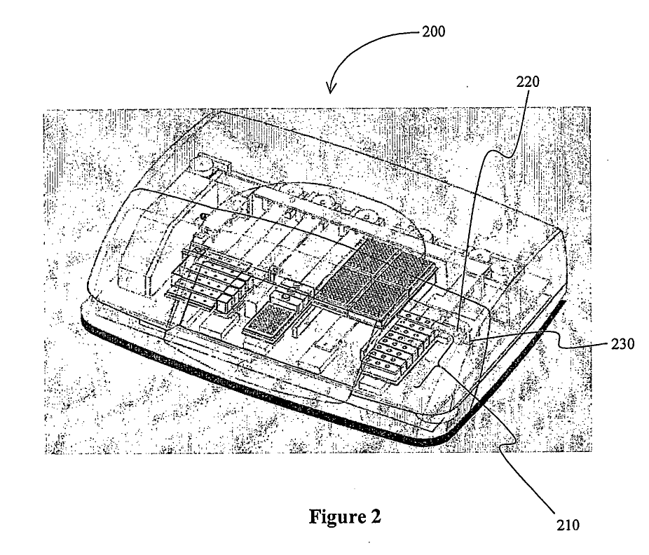

In an especially preferred aspect of the inventive subject matter, the closing element is moved between a first and a second position by an actuator of the analytic device to allow opening and closing of the reagent compartment(s) in a fully automated manner. Consequently, it should be recognized that the closing element of the multi-reagent pack is actuated while the multi-reagent pack is disposed within the analytic device (i.e., entirely enclosed by the analytic device). However, in less preferred aspects, an operator may open (or even remove) the closing element before the test procedure commences. An exemplary analytic device 200 is depicted in FIG. 2, wherein a plurality of multi-reagent packs 210 are disposed within the analytic device and opened by actuator 220, and wherein the electronic interface 230 communicates with the read-write memory chip (not shown) of the multi-reagent pack. Exemplary analytic devices are described in our copending international patent application with the serial number PCT/US02/17006 (filed 29 May, 2002; supra).

Suitable materials for disposable multi-reagent packs include natural and synthetic polymers, metals, metal alloys, carbon fiber based materials, and all reasonable combinations thereof. However, it is generally preferred that the multi-reagent pack is predominantly fabricated from polyethylene, polystyrene, or other low-cost materials with at least some resistance to chemical degradation. Thus, contemplated multi-reagent packs may also be coated with a specific coating to impart a desirable physico-chemical property (e.g., carbon black for light-protection), or include auxiliary materials to impart a desirable physico-chemical property (e.g., aluminum filings for heat transfer).

With respect to the pre-filled reagents, it should be appreciated that the chemical composition and volume of the pre-filled reagents may vary considerably, and all known reagents for analytic test procedures are considered suitable for use herein. Consequently, suitable reagents will include aqueous reagents (e.g., buffers, enzyme substrate solutions, nucleic acid or polypeptide-containing solutions, etc), as well as non-aqueous reagents (e.g., scintillation solutions, lipophilic solvents, etc.). Viewed from another perspective, suitable reagents include all reagents that are employed in performing various functions for analytic test procedure, including buffers to adjust/maintain a pH, wash solutions to remove unbound and/or unreacted components, substrate-containing solutions to provide a quantifiable signal (e.g., fluorophore, chromophore, luminogenic substrate), quenching solutions, and calibration solutions to normalize a signal generated in a test procedure. Of course, it should still further be appreciated that a reagent compartment may also be empty to receive contaminated or otherwise problematic fluid (e.g., fluid that requires sterilization prior to disposal, radioactive waste fluid). In yet another example, suitable reagents may also include disinfectants and other reagents that are not directly associated with determination of an analyte. For example, bleach may be employed as a reagent to disinfect a biochip prior to disposal. Alternatively, a humectant may be included to at least partially control evaporation.

It is further contemplated that multi-reagent packs according to the inventive subject matter will include a memory chip that at least temporarily stores information related to the multi-reagent pack, a test procedure that employs the multi-reagent pack, and other information (infra). While read-only memory chips are not expressly excluded, it is generally preferred that the memory chip is a read-write memory chip. There are numerous read-write memory chips known in the art and all of such read-write memory chips are considered suitable for use herein. However, especially suitable read-write memory chips include non-volatile read-write memory chips. Depending on the particular information stored on the memory chip, it is contemplated that the capacity of such chips may vary considerably. For example, where a memory chip stores routines and calibration curves for executing a particular test procedure, capacities of 128 Kb and even higher are deemed suitable. On the other hand, where only relatively little information is stored and/or written on the chip, capacities of equal or less than 32 Kb are contemplated.

In an especially preferred aspect of the inventive subject matter, the read-write memory chip will provide to the analytic device multi-reagent pack specific information, reagent-specific information, test-specific information, a locking code and/or a chronologic information, while the analytic device provides to the read-write memory chip at least one of a reagent-specific information, a locking code, and a chronologic information. For example, where quality control or other manufacture relevant information is desired by the operator of the analytical device (or by a person other than the operator via a data transfer element), it is contemplated that suitable multi-reagent pack specific information may comprise an individual identifier of the multi-reagent pack, a manufacture date of the multi-reagent pack, and/or shelf-life information of the first and/or second pre-filled reagents. In another example, contemplated multi-reagent pack specific information may also include a list of test procedures available for the first and second pre-filled reagents in the multi-reagent pack, or a manual or check-list of for the test procedure that employs the multi-reagent pack. In still further contemplated aspects, the multi-reagent pack specific information may include an operational and/or environmental parameter with specific significance to the multi-reagent pack (e.g., recorded use history, recommended storage conditions, recorded storage history, etc.).

Similarly, it is contemplated that the chip may also provide and/or receive reagent-specific information, wherein especially contemplated reagent-specific information comprises chemical composition and/or positional information of the first and second pre-filled reagents in the multi-reagent pack. Further examples of reagent specific information include the fill date, the original fill volume, and/or the remaining volume of the first and/or second pre-filled reagents during any time of use of the multi-reagent pack.

Where desired, the chip may also receive and/or provide the test-specific information. For example, test-specific information may include a test procedure that uses at least one of the first and second pre-filled reagents, or a reference code to the test procedure (wherein the reference code activates the test procedure stored in the analytical device). In still further contemplated examples of suitable test-specific information, and especially where the supplier desires control over the unauthorized refill, it is contemplated that the chip may receive from or provide to the analytic device a maximum allowed number of test procedures that uses the multi-reagent pack.

For example, the chip of a previously unused multi-reagent pack designed for 20 test procedures provides to the analytic device the information that no test has previously been performed, or that 20 tests using this multi-reagent pack are available. Once the analytic device completed a specific number of test procedures, the analytic device writes to the chip that either a specific number of tests has been performed, or adjusts a count-down setting accordingly to inform the device prior to the next use that 20 minus the specific number of tests are still available, or that a particular number of tests as indicated by the count-down setting are still available. Of course, it should be appreciated that similar data exchange and/or data modification can be performed with all data stored/provided by the chip.

In yet another preferred aspect of the inventive subject matter, it is contemplated that the chip may also include calibration data, and it is especially preferred that such calibration data will pertain to the first and/or second reagent in the multi-reagent pack. For example, where the first reagent comprises an optically detectable label, a previously established calibration curve using such first reagent may be written to the chip by the manufacturer to account, e.g., for deviations in concentration of the label. Alternatively, or additionally, calibration data may also be employed in conjunction with other data stored on the chip. For example, where the chip receives/provides chronologic information (e.g., cumulative time during which the closing element was opened for a specific reagent, or date/time of first use, or time elapsed since last use, etc.), test results may be normalized using such information. Such calibration is particularly advantageous where the reagent is prone to degradation once the container is opened, or decays over time.

Moreover, where desirable, the chip may also include a locking code that prohibits further use of the multi-reagent pack. For example, where use of the reagent pack is limited to selected operators, the analytic device may compare a locking code with the locking code previously stored on the chip (e.g., the locking code may be changed or erased by the operator using a password). In another example, the analytic device may write a locking code onto the chip to prevent further use of the multi-reagent pack in response to a condition of one or more of the reagents. For example, where the multi-reagent pack is designed for a specific test procedure and where the chip provides information to the analytic device that at least one reagent is not present in sufficient quantity for that test, the analytic device may prevent further use by writing a locking code to the chip. Alternatively, where at least one of the reagents is employed as an internal control for the test procedure to ascertain integrity of the reagents, the analytic device may prevent further use by writing a locking code to the chip where the analytic device detects less than desirable reagent quality.

While in numerous aspects of the present inventive subject matter various data are exchanged between the read-write chip and the analytic device and/or modified by the analytic device (or even by the read-write chip), it should also be recognized that a data transfer interface may be employed in the analytic device to provide to and/or receive data from the read-write memory chip (e.g., suitable data transfer interfaces include telephone, DSL, or cable modems, wireless networks, and hubs coupled to local networks).

For example, in one particularly preferred aspect, the data transfer interface is electronically coupled to a supplier that receives data from the read-write memory chip and/or the analytic device to initiate delivery of another multi-reagent pack, or to forecast demand for at least one of the first and second pre-filled reagents. Alternatively, the data transfer interface may be employed to troubleshoot a particular malfunction or other problem that an operator can not address locally. In such an instance, a supplier will electronically connect to the multi-reagent pack and/or analytic device and identify the condition of the analytic device (e.g., the analytic device or chip may create a failure code that can be read by the supplier).

Therefore, in a particularly preferred aspect of the inventive subject matter, the inventors contemplate that a multi-reagent pack has a first pre-filled reagent and a second pre-filled reagent, and that the multi-reagent pack further comprises a read-write memory chip that provides calculated volume information of the first and second pre-filled reagents to an analytic device, wherein the calculated volume information is generated by the analytic device using previous consumption of the first and second pre-filled reagent of the multi-reagent pack in the analytic device, and wherein the calculated volume information is written by the analytic device onto the read-write memory chip.

For example, a previously unused multi-reagent pack may provide the analytic device with information that volume of the first reagent is 50 ml. After a test procedure is performed using 5 ml of the first reagent, the analytic device will then modify the information on the chip such that when the multi-reagent pack is used in a second test procedure, the chip will provide the analytic device with information that volume of the first reagent is now 45 ml (e.g., which can be done by overwriting the 50 ml information, or by providing use information that is then computed by the analytic device). Such calculations may advantageously be used to prevent an unintended test interruption or other undesired situation in which a test procedure is started with insufficient reagent volume. For example, where a validation routine (e.g., performed on the analytic device) determines that insufficient reagent is present, the analytic device generates a locking code that prevents further use of the multi-reagent pack in the analytic device (e.g., the locking code may be written by the analytic device onto the read-write memory chip, or may be stored in the analytic device in association with a unique multi-reagent pack identifier code). Thus, viewed from another perspective, the inventors contemplate that the analytic device is programmed to perform a test procedure, wherein a validation sequence calculates reagent requirements for the first and second pre-filled reagents, and wherein the test procedure is not performed if the reagent requirements for the first and second pre-filled reagents are greater than the calculated volume information of the first and second pre-filled reagents.

Therefore, in a still further especially preferred aspect, the inventors contemplate a multi-reagent pack with a first pre-filled reagent and a second pre-filled reagent, wherein the multi-reagent pack further comprises a read-write memory chip that provides calibration data specific to at least one of the first and second pre-filled reagents. Moreover, the read-write memory chip in such devices may advantageously further provide to the analytic device (a) a protocol for a test procedure using the first and second pre-filled reagents or (b) an identifier that initiates a test procedure using the first and second pre-filled reagents. Of course, and as already discussed above, suitable analytic devices may further comprise a data transfer interface that is electronically coupled to a supplier, and wherein the supplier provides a data upgrade to at least one of the read-write memory chip and the analytic device (e.g., the data upgrade comprises corrected calibration data or a modified protocol for the test procedure using the first and second pre-filled reagents).

Especially contemplated analytic devices may further include automatic pipettors, detectors, and sample processing platforms to form an integrated analytic device. Particularly preferred sample processing platforms contemplated in conjunction with the teachings presented herein include those described in our co-pending international patent application with the title “Integrated Sample Processing Platform”, filed May, 28, 2003, which is incorporated by reference herein. Particularly preferred optical detectors contemplated in conjunction with the teachings presented herein include those described in our co-pending international patent application with the title “Microarray Detector and Methods”, filed May, 28, 2003, which is incorporated by reference herein. Particularly preferred automatic pipettors contemplated in conjunction with the teachings presented herein include those described in our co-pending international patent application with the title “Level-Controlled Pipette For Automated Analytic Devices”, filed May, 28, 2003, which is incorporated by reference herein.

Thus, specific embodiments and applications of improved reagent packs have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A disposable multi-reagent pack comprising:
a housing that includes a first compartment and a second compartment;
wherein the first and second compartments include a first pre-filled reagent and a second pre-filled reagent, respectively;
a closing element coupled to the housing and movable between a first and a second position, wherein the closing element is moved between the first and second position by an actuator when the multi-reagent pack is disposed within the analytic device, and wherein at least one of the first and second compartments is accessible to a pipette when the closing element is in the second position;
a read-write memory chip that provides to the analytic device at least one of a multi-reagent pack specific information, a reagent-specific information, a test-specific information, a locking code and a chronologic information; and
wherein the analytic device provides to the read-write memory chip at least one of a reagent-specific information, a locking code, and a chronologic information.

2. The disposable multi-reagent pack of claim 1 wherein the multi-reagent pack specific information comprises at least one of an individual identifier of the multi-reagent pack, a manufacture date of the multi-reagent pack, a list of test procedures available for the first and second pre-filled reagents, an environmental parameter, and a shelf-life of the at least one of the first and second pre-filled reagents.

3. The disposable multi-reagent pack of claim 1 wherein the reagent-specific information comprises at least one of a chemical composition of the first and second pre-filled reagents, positional information of the first and second pre-filled reagents, a fill date of at least one of the first and second pre-filled reagents, an original fill volume of at least one of the first and second pre-filled reagents, and a remaining volume of at least one of the first and second pre-filled reagents.

4. The disposable multi-reagent pack of claim 1 wherein the test-specific information comprises at least one of a test procedure that uses at least one of the first and second pre-filled reagents, a maximum allowed number of test procedures that uses the multi-reagent pack, calibration data, and a locking code.

5. The disposable multi-reagent pack of claim 1 wherein the chronologic information comprises cumulative time during which the closing element was in the second position, or time elapsed since first use of the multi-reagent pack.

6. The disposable multi-reagent pack of claim 1 wherein the locking code prohibits further use of the multi-reagent pack or can be erased using a user password.

7. The disposable multi-reagent pack of claim 1 wherein the analytic device further comprises a data transfer interface that is electronically coupled to a supplier that receives data from at least one of the read-write memory chip and the analytic device.

8. The disposable multi-reagent pack of claim 7 wherein the data is employed to initiate delivery of another multi-reagent pack or to forecast demand for at least one of the first and second pre-filled reagents.

9. The disposable multi-reagent pack of claim 1 wherein the housing includes a third compartment and a fourth compartment, and wherein the first and second pre-filled reagents are selected from the group consisting of a spectroscopically detectable agent, a fluorometrically detectable agent, a luminometrically detectable agent, and a radiometrically detectable agent, a nucleic acid, a polypeptide, and a buffer.

10. A multi-reagent pack with a first pre-filled reagent and a second pre-filled reagent, the multi-reagent pack further comprising a read-write memory chip that provides calculated volume information of the first and second pre-filled reagents to an analytic device, wherein the calculated volume information is generated by the analytic device using previous consumption of the first and second pre-filled reagent of the multi-reagent pack in the analytic device, and wherein the calculated volume information is written by the analytic device onto the read-write memory chip.

11. The multi-reagent pack of claim 10 wherein the calculated volume information is further calculated by the analytic device using an initial volume information provided by the read-write memory chip.

12. The multi-reagent pack of claim 10 wherein the analytic device generates a locking code that prevents further use of the multi-reagent pack in the analytic device.

13. The multi-reagent pack of claim 12 wherein the locking code is written by the analytic device onto the read-write memory chip.

14. The multi-reagent pack of claim 10 wherein the analytic device is programmed to perform a test procedure, wherein a validation sequence calculates reagent requirements for the first and second pre-filled reagents, and wherein the test procedure is not performed if the reagent requirements for the first and second pre-filled reagents are greater than the calculated volume information of the first and second pre-filled reagents.

15. The multi-reagent pack of claim 10 wherein the analytic device further comprises a data transfer interface that is electronically coupled to a supplier that receives data from at least one of the read-write memory chip and the analytic device.

16. The multi-reagent pack of claim 15 wherein the data is employed to initiate delivery of another multi-reagent pack or to forecast demand for at least one of the first and second pre-filled reagents.

17. A multi-reagent pack with a first pre-filled reagent and a second pre-filled reagent, the multi-reagent pack further comprising a read-write memory chip that provides calibration data specific to at least one of the first and second pre-filled reagents.

18. The multi-reagent pack of claim 17 wherein the read-write memory chip further provides to the analytic device (a) a protocol for a test procedure using the first and second pre-filled reagents or (b) an identifier that initiates a test procedure using the first and second pre-filled reagents.

19. The multi-reagent pack of claim 18 wherein the analytic device further comprises a data transfer interface that is electronically coupled to a supplier, and wherein the supplier provides a data upgrade to at least one of the read-write memory chip and the analytic device.

20. The multi-reagent pack of claim 19 wherein the data upgrade comprises corrected calibration data or a modified protocol for the test procedure using the first and second pre-filled reagents.
